(19) United States
(12) Patent Application Publication
(10) Pub. No.: US 2023/0117215 A1
(43) Pub. Date: Apr. 20, 2023

(54) METHOD FOR IDENTIFYING PRESTRESS FORCE IN SINGLE-SPAN OR MULTI-SPAN PCI GIRDER-BRIDGES

(71) Applicant: National Applied Research Laboratories, Taipei City (TW)

(72) Inventors: Marco BONOPERA, Taipei City (TW); Kuo-Chun CHANG, Taipei City (TW); Chung-Che CHOU, Taipei City (TW)

(21) Appl. No.: 17/538,038

(22) Filed: Nov. 30, 2021

Related U.S. Application Data
(60) Provisional application No. 63/257,315, filed on Oct. 19, 2021.

(51) Int. Cl. G01N 3/20; G01N 3/06; G01N 29/04; G01N 33/38

(52) U.S. Cl. CPC G01N 3/20; G01N 3/06; G01N 29/04; G01N 33/383; G01N 2203/0023; G01N 2203/06; G01N 2291/0232

(57) ABSTRACT

A method for identifying prestress force in single-span or multi-span PCI girder-bridges is provided. The method includes non-destructive steps for obtaining a set of parameters of the PCI girder-bridge under investigation, and combines various analyses to identify the change of prestress force. Therefore, the losses of prestress force are tracked and predicted. The method does not cause structural damages along the PCI girder-bridge, and the cost of the identification is significantly decreased.

### CROSS REFERENCE TO RELATED APPLICATION

This application claims the benefits of the U.S. Provisional Application Ser. No. 63/257,315, filed on Oct. 19, 2021, the subject matter of which is incorporated herein by reference.

### BACKGROUND OF THE INVENTION

### 1. Field of the Invention

The present invention relates to a method for identifying prestress force in single-span or multi-span Prestressed Concrete (PC) I girder-bridges; particularly, to a low-cost experimental method for solving this identification problem in single-span or multi-span PCI girder-bridges.

### 2. Description of Related Art

PCI girder-bridges are widely built worldwide. The most common are single-span, two-span, and three-span with a parabolic steel tendon (P), as illustrated in FIG. 1, FIG. 2, and FIG. 3, respectively, with their schematic and cross-sectional view (A-A). The serviceability and safety of single or multi-span PCI girder-bridges depend on the effective prestress force Nₓ. Therefore, it is very important to determine the prestress losses of PCI bridges at certain periods. Currently, there are many methods which were developed for identifying prestress losses in PCI bridges. Particularly, their predicted prestress losses were usually lower than those effective. Difficulties in obtaining accurate identifications are related to factors including assumptions about the prestressing systems and long-term phenomena, such as degradation processes, tendon relaxation, concrete creep and shrinkage, and environmental parameters. In general, such methods are very conservative. Thus, it remains impossible to correctly reflect the true condition of the PCI bridge under investigation. That is, the measurement of prestress losses in PCI bridges is very important. However, the existing methods are principally divided into destructive and dynamic non-destructive approaches. Those destructive are quite precise but cause significant structural damages along the PCI bridges. On the other hand, the dynamic nondestructive ones are unsuitable because a change in prestress force does not significantly influence the PCI bridges' vibration response. This makes the fundamental frequency an uncertain indicator of prestress losses. Consequently, those dynamics cannot furnish precise predictions.

Accordingly, a novel method for identifying the existing prestress force in PCI girder-bridges is needed. Especially, the identification based on static vertical deflections was preliminarily proved to be a reliable emerging technique and with a not significant structural impact (Bonopera M., Chang K.-C., Chen C.-C., Sung Y-C., Tullini N. Feasibility study of prestress force prediction for concrete beams using second-order deflections. International Journal of Structural Stability and Dynamics, 2018, 18(10), 1850124). Measured static vertical deflections indicate the changes which occur in the structural geometry due to prestress losses under equilibrium conditions, in turn, caused by the combined effects of tendon relaxation; concrete creep and shrinkage; temperature; and relative humidity. That is, a more reliable identification method can further be developed using static vertical deflections.

### SUMMARY OF THE INVENTION

A method for identifying prestress force in single or multi-span PCI girder-bridges is provided refer to FIG. 4 to FIG. 6, wherein the method includes the steps of: (A) obtaining a total length (L) with a tolerance of 1 mm and a first-order fundamental frequency (f_{1,I}) with a tolerance of 0.01 Hz of a PCI girder-bridge under investigation, and calculating or measuring its initial tangent Young's modulus with a tolerance of 1 MPa (E_{c, t} or E_{exp,c, t}), and the corresponding cross-sectional second moment of area (I_{1,I}) with a tolerance of 1 mm⁴; (B) performing a three-point bending test through a vertical load (F) with a tolerance of 0.1 kN for measuring the static vertical deflection at the PCI girder-bridge's midspan (v_{tot,mid}) with a tolerance of 0.01 mm, and a loading parameter (ψ); (C) calculating the non-dimensional prestress force (nₐ) by equation (I):

$${n}_{a} = {π}^{2} ( 1 - \frac{ψ}{χ {v}_{tot , mid}} ) {x}^{2} , \qquad ( I )$$

wherein x is 1 and χ is 48 when the PCI girder-bridge is a single span of length L (FIG. 4); x is 2 and χ is 534.26 when the PCI-girder-bridge is an equidistant two-span of length L (FIG. 5); x is 3 and χ is 2356.35 when the PCI-girder-bridge is an equidistant three-span of length L (FIG. 6); and

(D) determining the prestress force (Nₐ) by an equation (II):

$${N}_{a} = \frac{{E}_{exp , c , t} {I}_{1 , I}}{{L}^{2}} {n}_{a} . \qquad ( II )$$

In addition, the method for identifying prestress force in single or multi-span PCI girder-bridges is executed following the flow charts illustrated in FIG. 7 and FIG. 8.

Refer to FIG. 9, in one embodiment of step (A), when the initial tangent Young's modulus (E_{exp,c, t}) is measured, and the total self-mass per unit length (m_{PCI+d}=m_{PCI}+m_{d}) is known (m_{PCI} and m_{d} are the total self-mass per unit length of PCI girder-bridge and deck, respectively), the first-order fundamental frequency (f_{1,I}) is evaluated.

In one embodiment, in step (A), when the PCI girder-bridge is the single-span of length L, the first-order fundamental frequency (f_{1,I}) is calculated by the analytical solution proposed by Song (Song 2000, Dynamics of Highway Bridges. Beijing, China: China Communications Press, 113-120. Chapter 1), whereas the cross-sectional second moment of area (I_{1,I}) is calculated by equation (III-1) based on Euler-Bernoulli theory:

$${I}_{1 , I} = \frac{4 {f}_{1 , I}^{2} {m}_{P C I + d} {L}^{4}}{{π}^{2} {E}_{exp , c , t} g} , \qquad ( III - 1 )$$

wherein g=9.81 m/s².

In one embodiment, the first-order fundamental frequency (f_{1,I}) is calculated by the analytical solution shown in equation (2):

$${f}_{1 , I} = \sqrt{\frac{{E}_{exp , c , t} {I}_{tot , mid} {π}^{5} + 32 λ {f}_{t} {L}^{2}}{2 {m}_{P C I + d} π {L}^{4}}} , \qquad ( 2 )$$

wherein I_{tot,mid} is the cross-sectional second moment of area of the PCI girder-bridge's midspan (concrete and tendon); λ is a first-order coefficient, whereas fₜ is the deflected shape of the parabolic tendon (fₜ=e₂+e₁; wherein e₁ and e₂ are eccentricities of parabolic tendon).

In one embodiment, the first-order coefficient λ is evaluated by equation (3):

$$λ = \frac{{E}_{t} {A}_{t}}{{L}_{t}} [ \frac{16 {f}_{t}}{π L} - \frac{2 {L}^{3}}{{E}_{e x p , c , t} {I}_{tot , mid} {π}^{3}} ( - {m}_{P C I + d} ) ] , \qquad ( 3 )$$

wherein Eₜ is the Young's modulus of parabolic tendon; Aₜ is its cross-sectional area, whereas Lₜ is its effective length.

In detail, the initial tangent Young's modulus of concrete at the time of testing (E_{exp,c, t}) is evaluated using Model B4 (Bažant Z P, Jirásek M, Hubler M H, Carol I. RILEM draft recommendation: TC-242-MDC multi-decade creep and shrinkage of concrete: material model and structural analysis. Model B4 for creep, drying shrinkage and autogenous shrinkage of normal and high-strength concretes with multi-decade applicability. Mater. Struct. 2015; 48(4):753-70) as follows:

E_{exp,c,t}=15,000\sqrt{f_{c,aver,t}}.

in which f_{c,aver, t} is the mean compressive drilled cylinder strength of concrete measured by compression tests at the time of testing.

In Taiwan, E_{exp,c, t} can be evaluated using Model B4-TW (Hu W H, Liao W C. Study of prediction equation for modulus of elasticity of normal strength and high strength concrete in Taiwan. J. Chin. Inst. Eng. 2020; 43(7):638-47) as follows:

E_{exp,c,t}=12,000\sqrt{f_{c,aver,t}}.

In one embodiment, in step (A), when the PCI girder-bridge is single-span or multi-span, its first-order fundamental frequency (f_{1,I,FE}) is calculated using the Finite Element (FE) model proposed by Jaiswal (2008) for PC girder-bridges with a parabolic bonded tendon (Jaiswal 2008, Effect of prestressing on the first flexural natural frequency of beams, Structural Engineering and Mechanics, 28(5):515-524). The cross-sectional second moment of area (I_{1,I,FE}) is consequently determined based on the Euler-Bernoulli theory.

Referring FIG. 10, when the PCI girder-bridge is the single-span of length L, the cross-sectional second moment of area (I_{1,I,FE}) is calculated by equation (III-2-1):

$${I}_{1 , I , FE} = \frac{4 {f}_{1 , I , FE} {}^{2} {m}_{t o t} {L}^{4}}{{π}^{2} {E}_{exp , c , t} g} . \qquad ( III - 2 - 1 )$$

Refer to FIG. 11, when the PCI girder-bridge is the equidistant two-span of length L, the cross-sectional second moment of area (I_{1,I,FE}) is calculated by equation (III-2-2):

$${I}_{1 , I , FE} = \frac{{f}_{1 , I , FE} {}^{2} {m}_{t o t} {L}^{4}}{4 {π}^{2} {E}_{exp , c , t} g} . \qquad ( III - 2 - 2 )$$

Refer to FIG. 12, when the PCI girder-bridge is the equidistant three-span of length L, the cross-sectional second moment of area (I_{1,I,FE}) is instead calculated by equation (III-2-3):

$${I}_{1 , I , FE} = \frac{{f}_{1 , I , FE} {}^{2} {m}_{t o t} {L}^{4}}{20.25 {π}^{2} {E}_{exp , c , t} g} . \qquad ( III - 2 - 3 )$$

In equations (III-2-1) to (III-2-3), g=9.81 m/s²; mₜₒₜ is the PCI girder-bridge's total self-mass per unit length which, in turn, is given by the sum of total self-mass per unit length of PCI girder-bridge m_{PCI} (concrete and rebars), parabolic tendon mₜ and deck m_{d} (m_{PCI}+mₜ+m_{d}). I_{1,I,FE} is regarded as the cross-sectional second moment of area (I_{1,I}) for subsequent steps. The corresponding eccentricities of parabolic tendon e₁ and e₂ (FIG. 10) or e₁, e₂ and e₃ must be considered in the FE models (FIG. 11 and FIG. 12).

In one embodiment, in step (A), when the cross-sectional second moment of area (I_{1,I}) is unknown, and when the PCI girder-bridge is single-span or multi-span, the first-order fundamental frequency (f_{1,exp}) is measured through free bending vibration tests. The cross-sectional second moment of area (I_{1,I,exp}) is consequently estimated based on the Euler-Bernoulli theory. In fact, since free bending vibrations are very small, the influence of prestress force on the dynamics of PCI girder-bridges is negligible (Bonopera M., Chang K.-C., Chen C.-C., Sung Y.-C., Tullini N. Prestress force effect on fundamental frequency and deflection shape of PCI beams. Structural Engineering and Mechanics, 2018, 67(3), 255-265).

Refer to FIG. 13, when the PCI girder-bridge is the single-span of length L, the cross-sectional second moment of area (I_{1,I,exp}) is calculated by equation (III-3-1):

$${I}_{1 , I , FE} = \frac{4 {f}_{1 , exp} {}^{2} {m}_{t o t} {L}^{4}}{{π}^{2} {E}_{exp , c , t} g} . \qquad ( III - 3 - 1 )$$

Refer to FIG. 14, when the PCI girder-bridge is the equidistant two-span of length L, the cross-sectional second moment of area (I_{1,I,exp}) is calculated by equation (III-3-2):

$${I}_{1 , I , exp} = \frac{{f}_{1 , exp} {}^{2} {m}_{t o t} {L}^{4}}{4 {π}^{2} {E}_{exp , c , t} g} . \qquad ( III - 3 - 2 )$$

Refer to FIG. 15, when the PCI girder-bridge is the equidistant three-span of length L, the cross-sectional second moment of area (I_{1,I,exp}) is instead calculated by equation (III-3-3):

$${I}_{1 , I , exp} = \frac{{f}_{1 , exp} {}^{2} {m}_{t o t} {L}^{4}}{20.25 {π}^{2} {E}_{exp , c , t} g} . \qquad ( III - 3 - 3 )$$

In equations (III-3-1) to (III-3-3), g=9.81 m/s²; mₜₒₜ is the PCI girder-bridge's total self-mass per unit length which, in turn, is given by the sum of total self-mass per unit length of PCI girder-bridge m_{PCI} (concrete and rebars), parabolic tendon mₜ and deck m_{d} (m_{PCI}+mₜ+m_{d}).

A calibrated cross-sectional second moment of area (I_{1,I,cal}) is consequently estimated by equation (IV):

I_{1,I,cal}=0.93×I_{1,I,exp} (IV),

wherein I_{1,I,cal} is regarded as the cross-sectional second moment of area (I_{1,I}) for subsequent steps.

In one embodiment, in step (B), the loading parameter (y) is measured by equation (V):

$${ψ}_{cal} = \frac{F {L}^{3}}{{E}_{e x p , c , t} {I}_{1 , I , ca1}} . \qquad ( V )$$

In detail, referring from FIG. 16 to FIG. 18, the vertical load F of three-point bending test in step (B) is determined considering the Euler-Bernoulli theory's assumptions, and assuming the first-order static vertical deflection at the PCI girder-bridge's midspan (v_{I,mid}) with a value between 4.50 and 7.00 mm. As a result, the formula for determining the vertical load (F) is equal to:

$$F = \frac{χ {v}_{I , mid} {E}_{c , t} {I}_{t o t , m i d}}{{L}^{3}} ,$$

wherein χ is 48 when the PCI girder-bridge is the single-span of length L as illustrated in FIG. 16; χ is 534.26 when the PCI-girder-bridge is the equidistant two-span of length L (FIG. 17); χ is 2356.35 when the PCI-girder-bridge is the equidistant three-span of length L (FIG. 18). I_{tot,mid} is the cross-sectional second moment of area of the PCI girder-bridge's midspan (concrete and tendon). E_{c, t} is the initial tangent Young's modulus of concrete evaluated at the time of testing. In the abovementioned equation, a static vertical deflection (v_{I,mid}) with a value higher than 5.00 mm is suggested.

When the design parameters of the PCI bridge are unknown, the aforementioned formula can adopt the cross-sectional second moment of area of the PCI girder-bridge under investigation I (concrete only) after measurement of dimensions in-situ. The initial tangent Young's modulus at the time of testing (E_{c, t}) can instead be evaluated using Model B4 as follows:

$${E}_{c , t} = {{E}_{c , 28} [ \frac{t}{4 + ( 6 / 7 ) t} ]}^{0.5} ,$$

wherein t is the time of testing in days of concrete curing, whereas the initial tangent Young's modulus at 28 days of curing (E_{c,28}) is evaluated as follows:

E_{c,28}=4,734\sqrt{f_{c,aver,28}},

wherein f_{c,aver,28} is the mean compressive cylinder strength at 28 days of concrete curing. In Taiwan, the initial tangent Young's modulus at 28 days of curing (E_{c,28}) is evaluated using Model B4-TW as follows:

E_{c,28}=3,831\sqrt{f_{c,aver,28}}.

In one embodiment of steps (C) and (D), performing the three-point bending test through a vertical load F for measuring the static vertical deflection at the midspan (v_{tot,mid}) of the single-span PCI girder-bridge (FIG. 4), and the corresponding loading parameter (ψ=FL³/E_{exp,c, t}I), it is possible to obtain the non-dimensional prestress force (nₐ) by equation:

$${n}_{a} = {π}^{2} ( 1 - \frac{ψ}{48 {v}_{tot , mid}} ) ,$$

wherein the vertical deflection (v_{tot,mid}) is given by the following expression after measurements v_{tot,mid}=v_{exp,1}−(v_{exp,0}/2)−(v_{exp,2}/2). The existing prestress force (Nₐ) is consequently identified by equation:

$${N}_{a} = \frac{{E}_{e x p , c , t} I}{{L}^{2}} {n}_{a} ,$$

wherein the cross-sectional second moment of area (J) is regarded as the cross-sectional second moment of area I_{1,I}, I_{1,I,FE} or I_{1,I,exp}, respectively. When the PCI girder-bridge is the equidistant two-span of total length L (FIG. 5), the equation becomes:

$${n}_{a} = 4 {π}^{2} ( 1 - \frac{ψ}{534.26 {v}_{tot , mid}} ) .$$

Conversely, when the PCI girder-bridge is the equidistant three-span of total length L (FIG. 6), the equation becomes:

$${n}_{a} = 9 {π}^{2} ( 1 - \frac{ψ}{2 , 356.35 {v}_{tot , mid}} ) .$$

The initial tangent Young's modulus of concrete at the time of testing (E_{c, t}) can also be evaluated analytically according to Model B4 or Model B4-TW based on the location of the PCI bridge.

### BRIEF DESCRIPTION OF THE DRAWINGS

FIG. 1 is a schematic view of a single-span PCI girder-bridge;

FIG. 2 is a schematic view of a two-span PCI girder-bridge;

FIG. 3 is a schematic view of a three-span PCI girder-bridge;

FIG. 7 is the first flow chart of evaluation of the cross-sectional second moment of area of one embodiment of the present invention;

FIG. 8 is the second flow chart of evaluation of the cross-sectional second moment of area of one embodiment of the present invention;

FIG. 9 is a schematic view of a single-span PCI girder-bridge of one embodiment of the present invention;

FIG. 10 is a schematic view of a single-span PCI girder-bridge of one embodiment of the present invention;

FIG. 11 is a schematic view of a two-span PCI girder-bridge of one embodiment of the present invention;

FIG. 12 is a schematic view of a three-span PCI girder-bridge of one embodiment of the present invention;

FIG. 13 is a schematic view of a single-span PCI girder-bridge of one embodiment of the present invention;

FIG. 14 is a schematic view of a two-span PCI girder-bridge of one embodiment of the present invention;

FIG. 15 is a schematic view of a three-span PCI girder-bridge of one embodiment of the present invention;

FIG. 16 is a schematic view of a single-span PCI girder-bridge for determining the vertical load F of three-point bending test of one embodiment of the present invention;

FIG. 17 is a schematic view of a two-span PCI girder-bridge for determining the vertical load F of three-point bending test of one embodiment of the present invention;

FIG. 18 is a schematic view of a three-span PCI girder-bridge for determining the vertical load F of three-point bending test of one embodiment of the present invention;

FIG. 19 is a schematic view which shows the test layout of the single-span PC girder-bridge of the laboratory simulation of the present invention;

FIG. 20 shows the accelerations (A3, m/s²) measured at cross-section i=3 at 291 days of prestressing of one embodiment of the present invention;

FIG. 21 shows the fast Fourier transform of A3 of one embodiment of the present invention.

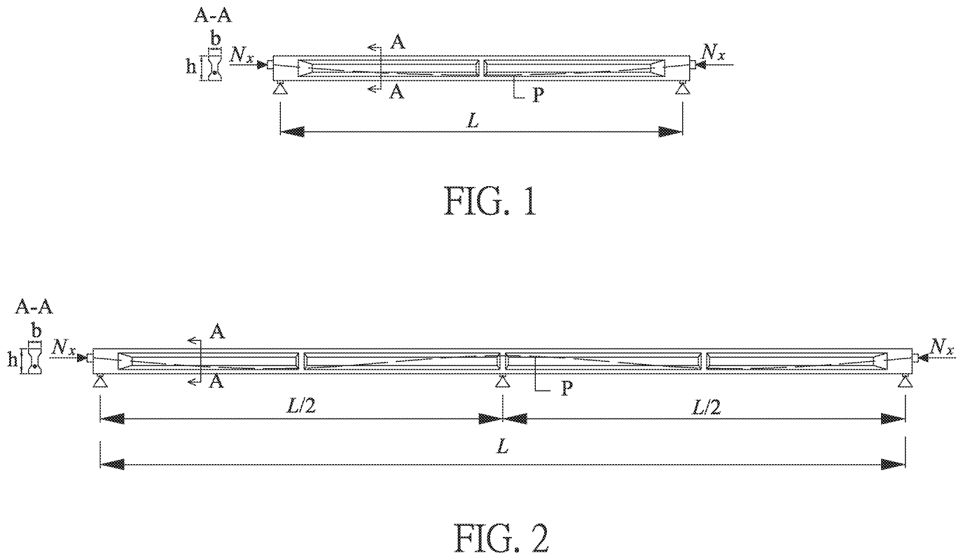

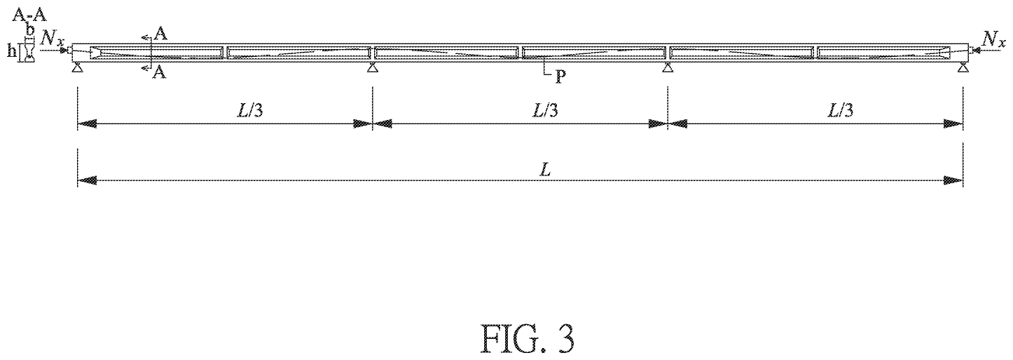

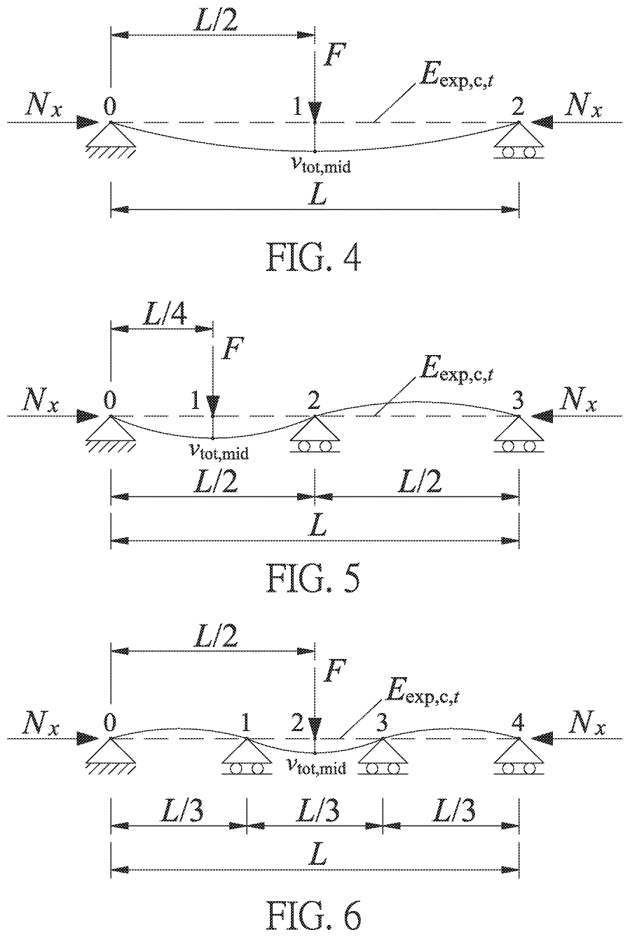

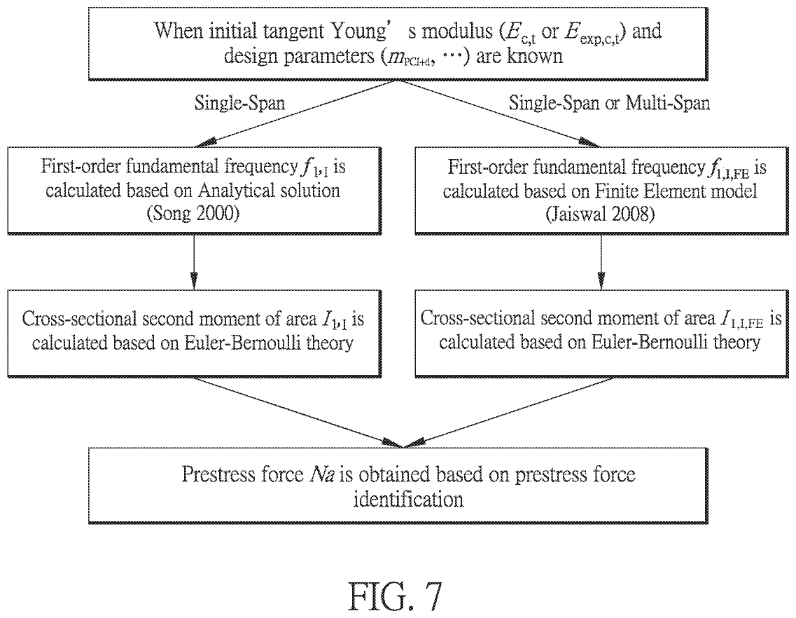

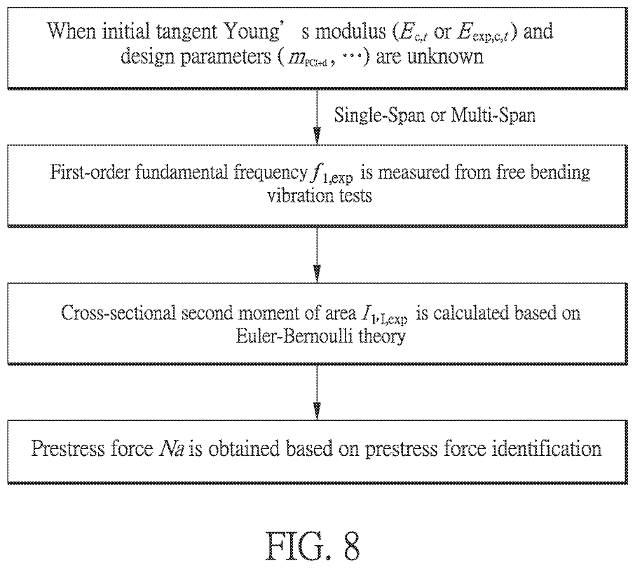

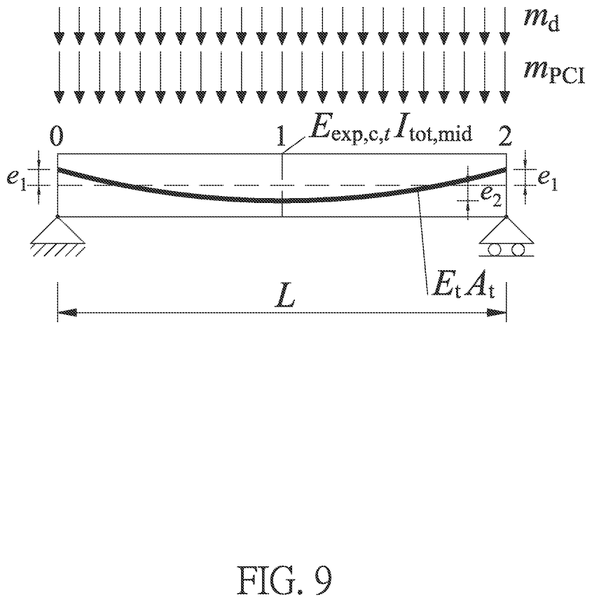

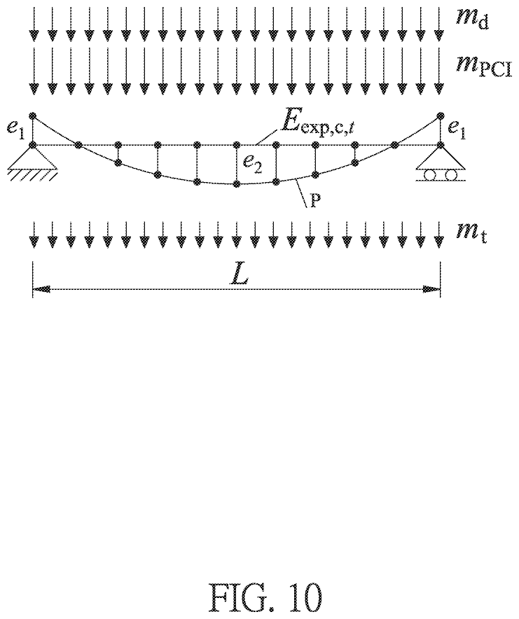

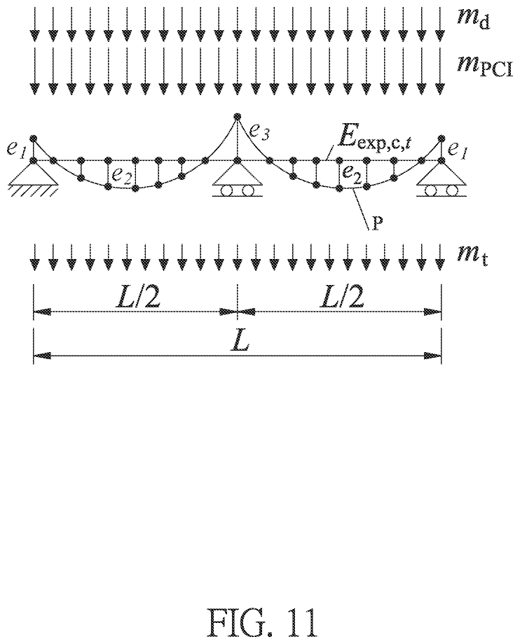

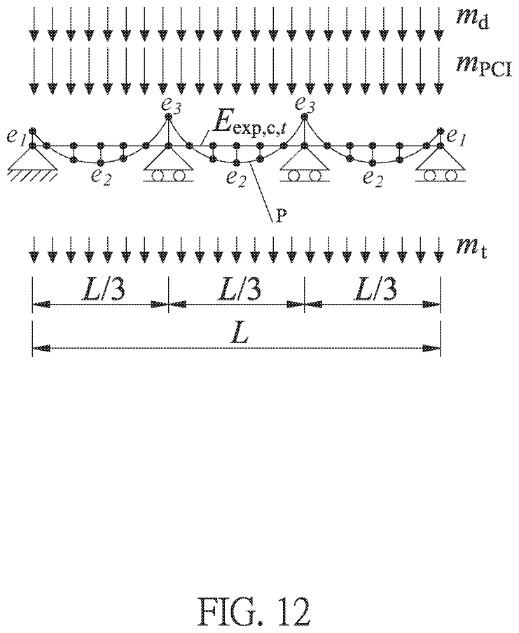

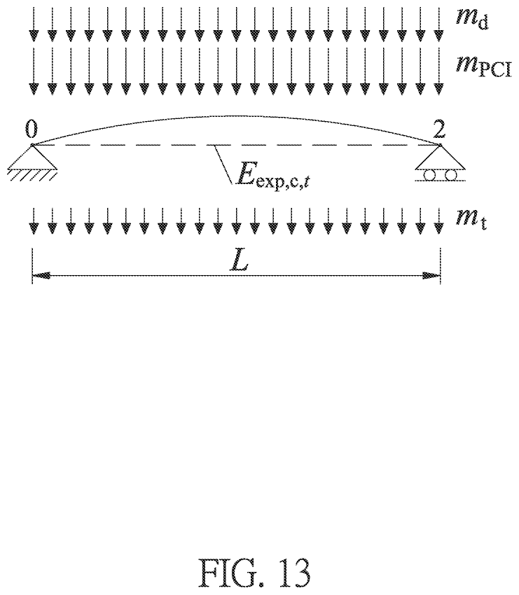

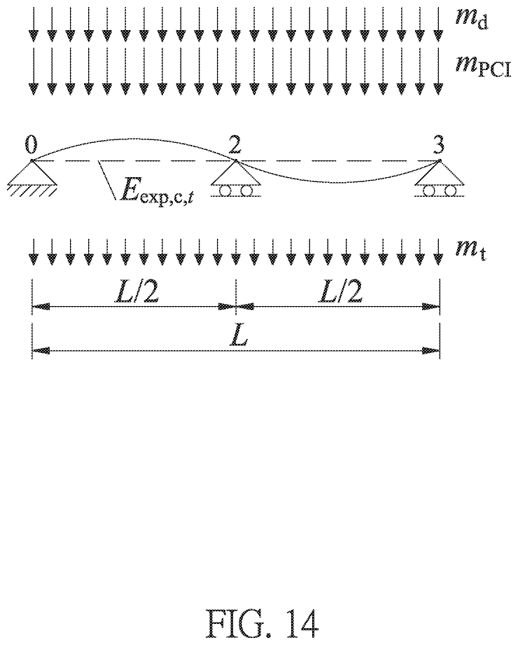

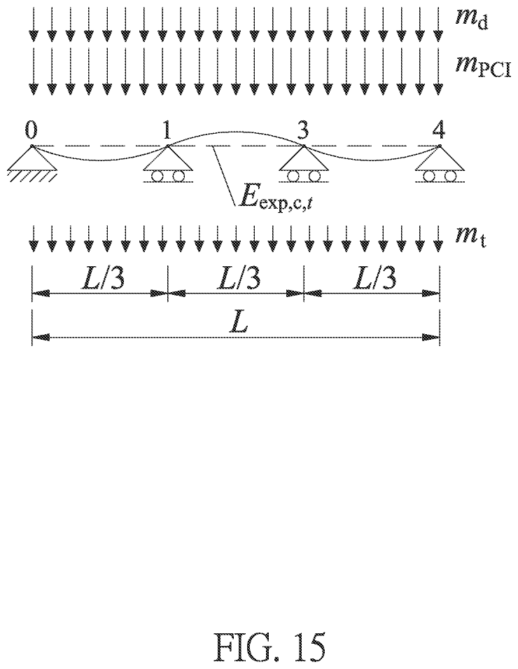

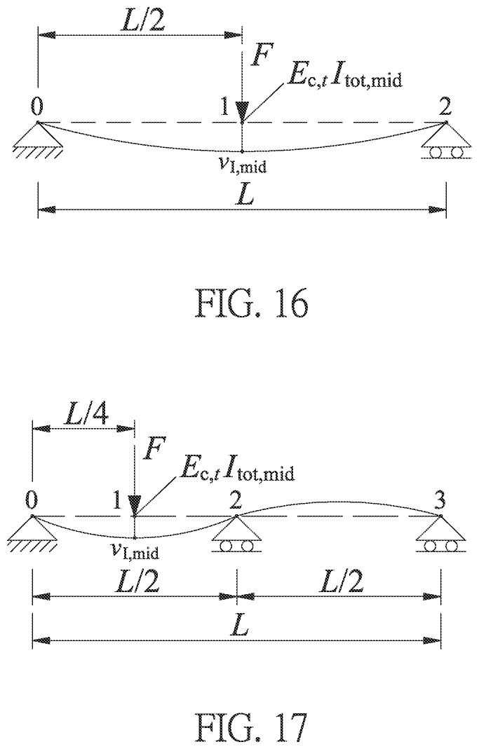

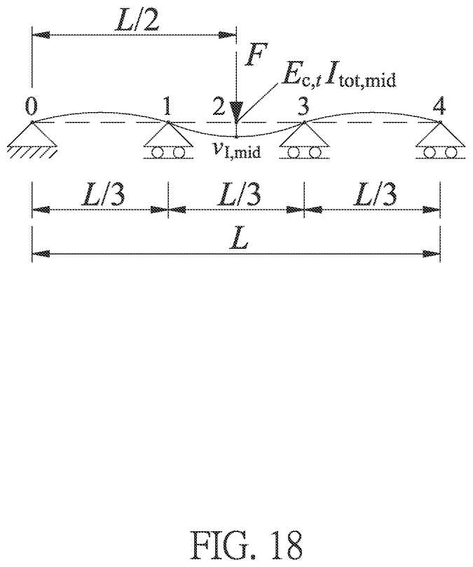

### DETAILED DESCRIPTION OF THE PREFERRED EMBODIMENT

[Single-Span PC Girder-Bridge Prototype]

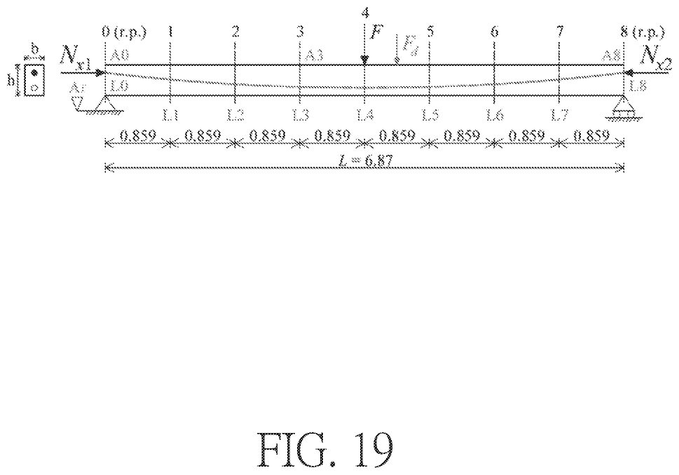

The PC girder-bridge prototype was composed of a high-strength concrete made in Taiwan, and reinforced with steel rebars and stirrups with a unit weight (ρₛ) of ≈1.23 kN/m³. The concrete's unit weight was 22.90 kN/m³. As illustrated in FIG. 19, two pinned-end supports were arranged at its ends for a clear span (L) of 6,870 mm. The ultimate yield strength (σ_{uy}), Young's modulus (Eₜ), and unit weight of the strands (ρₜ) were 1,860 MPa, 200 GPa, and 76.65 kN/m³, respectively. The cross-sectional second moment of area of the PC girder-bridge's concrete-only section (I) was 1.2775×10⁹ mm⁴. The corresponding cross-sectional area (A) was 9.727×10⁴ mm². Furthermore, the cross-sectional area of the parabolic tendon (Aₜ) was 973 mm², whereas its effective length was Lₜ=[1+8/3×(fₜ/L)²]×L=6,886 mm.

[Measurement of Prestress Losses]

The PC girder-bridge prototype was positioned in a test rig (FIG. 19). At one of its ends, a hydraulic oil jack was used to create a prestress force (N_{0×,aver}) of ≈600 kN at an age of concrete of 127 days by pulling the parabolic tendon outwardly. A sensor was arranged at both ends to measure the prestress forces N_{0×1} and N_{0×2} caused by elastic shortening phenomena (Table 1). A mean prestress force (N_{0×,aver}) of 557 kN was then measured after 7 days of curing of cement mortar, with which the parabolic tendon was injected, that was at a concrete age of 134 days. At this time, the shortening prestress losses were at last of 7.2%. Notably, the end of the 7 days of curing of cement mortar was assumed as the initial time of prestressing. Refer to Table 1, the prestress forces (N_{0×,aver}) were subsequently measured at durations of 3, 8, 10, 15, 17, 24, 29, 31, 43, 45, 57, and 66 days.

TABLE 1

| Age of | Age of | | | | Prestress | | | | | | | | | | | |
|---|---|---|---|---|---|---|---|---|---|---|---|---|---|---|---|---|
| concrete | prestressing | N_{0×1} | N_{0×2} | N_{0x, aver} | losses | Nₓ₁ | Nₓ₂ | | F | v₁ | v₂ | v₃ | v₄ | v₅ | v₆ | v₇ |
| (days) | (days) | (kN) | (kN) | (kN) | (%) | (kN) | (kN) | N_{x, aver} | (kN) | (mm) | (mm) | (mm) | (mm) | (mm) | (mm) | (mm) |
| 127 | — | ~629 | ~571 | ~600 | — | — | — | — | — | — | — | — | — | — | — | — |
| 134 | 1 | 586 | 527 | 557 | −7.2 | — | — | — | — | — | — | — | — | — | — | — |
| 136 | 3 | 586 | 527 | 557 | −7.2 | — | — | — | — | — | — | — | — | — | — | — |
| 141 | 8 | 586 | 527 | 557 | −7.2 | — | — | — | — | — | — | — | — | — | — | — |
| 143 | 10 | 585 | 527 | 556 | −7.3 | — | — | — | — | — | — | — | — | — | — | — |
| 148 | 15 | 585 | 526 | 555 | −7.5 | — | — | — | — | — | — | — | — | — | — | — |
| 150 | 17 | 585 | 526 | 556 | −7.3 | — | — | — | — | — | — | — | — | — | — | — |
| 157 | 24 | 584 | 525 | 555 | −7.5 | — | — | — | — | — | — | — | — | — | — | — |
| 162 | 29 | 583 | 524 | 554 | −7.7 | — | — | — | — | — | — | — | — | — | — | — |
| 164 | 31 | 583 | 525 | 554 | −7.7 | — | — | — | — | — | — | — | — | — | — | — |
| 176 | 43 | 582 | 524 | 553 | −7.8 | — | — | — | — | — | — | — | — | — | — | — |
| 178 | 45 | 582 | 524 | 553 | −7.8 | — | — | — | — | — | — | — | — | — | — | — |
| 190 | 57 | 581 | 523 | 552 | −8.0 | — | — | — | — | — | — | — | — | — | — | — |
| 199 | 66 | 579 | 521 | 550 | −8.3 | — | — | — | — | — | — | — | — | — | — | — |
| 421 | 288 | 533 | 499 | 516 | −14.0 | 533 | 499 | 516 | 23.2 | 1.18 | 2.13 | 2.79 | 3.15 | 2.86 | 2.17 | 1.26 |
| | | | | | | | | | 34.2 | 1.76 | 3.16 | 4.15 | 4.65 | 4.25 | 3.23 | 1.87 |
| | | | | | | | | | 42.2 | 2.18 | 3.93 | 5.16 | 5.75 | 5.27 | 4.01 | 2.33 |
| 423 | 290 | 533 | 499 | 516 | −14.0 | 533 | 499 | 516 | 24.2 | 1.22 | 2.21 | 2.89 | 3.26 | 2.97 | 2.25 | 1.29 |
| | | | | | | | | | 33.2 | 1.68 | 3.06 | 4.01 | 4.50 | 4.11 | 3.10 | 1.78 |
| | | | | | | | | | 42.8 | 2.22 | 4.00 | 5.24 | 5.85 | 5.36 | 4.07 | 2.35 |
| | | | | | | | | | 51.4 | 2.63 | 4.80 | 6.33 | 7.04 | 6.45 | 4.89 | 2.81 |
| 424 | 291 | 532 | 498 | 515 | −14.2 | 532 | 498 | 515 | 26.5 | 1.36 | 2.44 | 3.21 | 3.55 | 3.27 | 2.48 | 1.43 |
| | | | | | | | | | 34.1 | 1.76 | 3.16 | 4.16 | 4.59 | 4.22 | 3.20 | 1.82 |
| | | | | | | | | | 42.1 | 2.18 | 3.93 | 5.20 | 5.72 | 5.26 | 4.00 | 2.25 |
| | | | | | | | | | 51.9 | 2.69 | 4.88 | 6.45 | 7.12 | 6.53 | 4.96 | 2.78 |

[Free Bending Vibration Tests]

Free vibrations were generated by breaking a series of steel rebars of a diameter of 8 mm which were installed near the PC girder-bridge's midspan. Its self-mass per unit length (m_{PCI}) was 2.392 kN/m (concrete+rebars). When the rebars ruptured, the PC girder-bridge was vertically excited by small unbalanced forces. Therefore, its vibrational response was measured along the strong axis. Vibration measurements were repeated thrice at prestressing durations of 288, 290, and 291 days, respectively. The average measurements of the applied prestress forces N_{0×1} and N_{0×2} for every test day were listed in Table 1.

[Three-Point Bending Tests]

A vertical load (F) of different values was applied by a transverse steel beam at the PC girder-bridge's midspan at prestressing durations of 288, 290, and 291 days. Displacement transducers were used to measure the static vertical deflections vᵢ, for i=0, . . . , 8 (FIG. 19). The results were reported in Table 1.

[Estimation of Young's Modulus]

The Young's modulus of the PC girder-bridge prototype was measured by compression tests, according to ASTM C 469/C 469M-14 (Annual Book of ASTM Standards 2016). The results were reported in Table 2. The mean compressive cylinder strength (f_{c,aver,28}) and the average chord Young's modulus (E_{exp,28}) at 28 days were 88 and 35,060 MPa, respectively (Table 2). The mean compressive strength (f_{c,aver,431}) and the average chord Young's modulus (E_{exp,431}) of the drilled cores at 431 days of concrete curing were instead 92 and 37,889 MPa, respectively, i.e., 4.5 and 8.1% higher than the corresponding values at 28 days.

In addition, the initial tangent Young's modulus of the high-strength concrete (E_{exp,c,431}) at 431 days was evaluated by equation (1), according to Model B4-TW, wherein the Young's modulus (E_{exp,c,431}) is expressed in kg/cm²:

E_{exp,c,431}=12,000\sqrt{f_{ck,aver,431}} (1)

TABLE 2

| Age of concrete t | N_{0x, aver} | f_{ck, aver, t} | E_{exp, c, t} |
|---|---|---|---|
| (days) | (kN) | (MPa) | (MPa) |
| 28 | — | 88 | 35,198 |
| 431 | 516 | 92 | 36,054 |

[Evaluation of the Cross-Sectional Second Moment of Area—Analytical Solution]

When the PCI girder-bridge is a single-span, its cross-sectional second moment of area (I_{1,I}) is determined by substituting the first-order fundamental frequency (f_{1,I}) into equation (III-1) based on the Euler-Bernoulli theory.

In equation (III-1), g=9.81 m/s². The fundamental frequency (f_{1,I}) is calculated by the analytical solution, which includes the following equations (2) and (3). I_{tot,mid} is the cross-sectional moment of area of the PC girder-bridge's midspan (concrete and tendon), which was assumed to be equal to 1.3261×10⁹ mm⁴, according to the design. λ is a first-order coefficient which is calculated by equation (3).

The effective cross-sectional second moment of area (I_{tot,mid}) and that obtained from the aforementioned procedure (I_{1,I}) were reported in Table 3. According to the results, the value of the cross-sectional second moment of area (I_{1,I}) evaluated by the analytical solution, and based on the Euler-Bernoulli theory, was reliable. Consequently, its use as parameter within the present invention was implemented in the subsequent calculations.

[Evaluation of the Cross-Sectional Second Moment of Area—Finite Element Model]

In the present embodiment, when the PCI girder-bridge is a multi-span, its fundamental frequency (f_{1,I,FE}) is determined according to the Finite Element (FE) model (Jaiswal O R, 2008, Effect of prestressing on the first flexural natural frequency of beams, Structural Engineering and Mechanics, 28(5):515-524). Its cross-sectional second moment of area (I_{1,I,FE}) is then determined by substituting the FE fundamental frequency (f_{1,I,FE}) into equation (III-2), which represents the first-order fundamental frequency of a single-span Euler-Bernoulli beam, wherein mₜₒₜ=(m_{PCI}+mₜ)=[m_{PCI}+(ρₜ×A₁)]=2.4666 kN/m.

The FE fundamental frequency (f_{1,I,FE}), cross-sectional second moment of area (I_{1,I,FE}), and effective cross-sectional second moment of area (I_{1,I}) obtained from the aforementioned procedure were also reported in Table 3. According to the results, the value of the cross-sectional second moment of area (I_{1,I}) evaluated by the analytical solution, and based on the Euler-Bernoulli theory, was reliable. Consequently, its use as a parameter within the present invention was implemented in the subsequent calculations.

[Evaluation of the Cross-Sectional Second Moment of Area—Experimental Method]

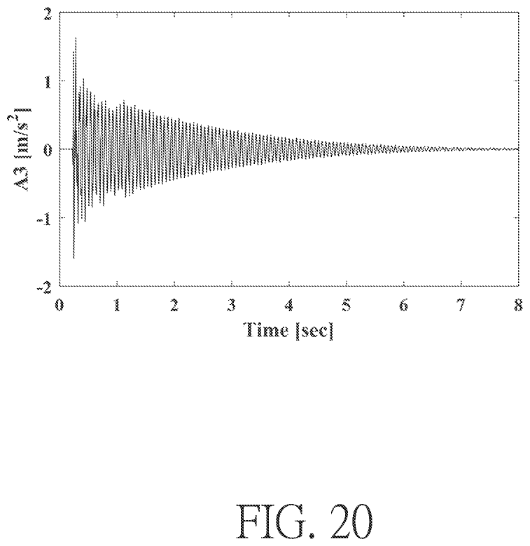

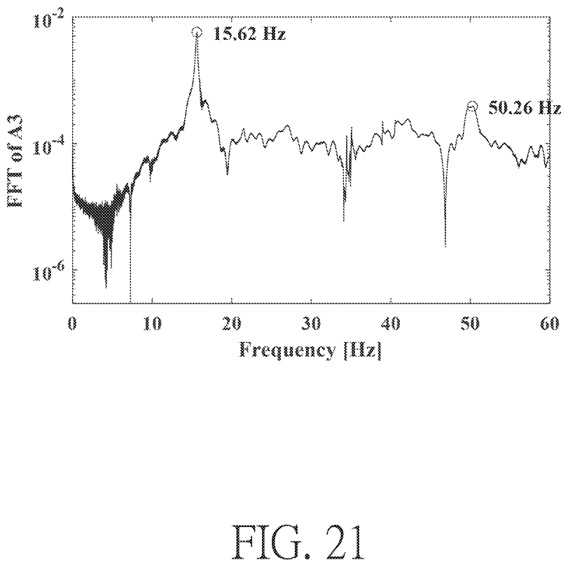

In the present embodiment, when the PCI girder-bridge is single-span or multi-span, and its design parameters are unknown, the flexural rigidity is estimated through free bending vibrations. In short, its first-order fundamental frequency (f_{1,exp}) is obtained using free bending vibration tests. The test results were shown in FIG. 20 and FIG. 21, wherein acceleration versus time for instrumented section A3 (m/s²) at 291 days of prestressing was shown in FIG. 20. The Fast Fourier transform for a block size of 65,536 samples, and using Peak Picking method, was instead shown in FIG. 21. The experimental fundamental frequencies (f_{1,exp}) provided by the seismometer A3 were 15.60, 15.60 and 15.62 Hz at 288, 290 and 291 days of prestressing, respectively. Next, the fundamental frequencies (f_{1,exp}) were substituted into equation (III-3), based on the Euler-Bernoulli theory, for the cross-sectional second moment of area (I_{1,I,exp}) of a single-span PCI girder-bridge.

In equation (III-3), g=9.81 m/s²; mₜₒₜ=(m_{PCI}+mₜ) is the total self-mass per unit length given by the sum of self-mass per unit length of PCI girder-bridge and that of parabolic tendon.

According to the results (Table 3), and based on the aforementioned calculations, when E_{exp,c,431}=36,054 MPa is brining into the equations, I_{1,I,exp} is 1.54285×10⁹ mm⁴ at 288 and 290 days, whereas is equal to 1.54680×10⁹ mm⁴ at 291 days of prestressing. Next, a calibrated cross-sectional second moment of area (I_{1,I,cal}) is obtained by the calibration equation (IV). The results of calibration were also reported in Table 3.

TABLE 3

| Age of | | | | | | | | |
|---|---|---|---|---|---|---|---|---|
| prestressing | f_{1, I} | I_{1, I} | I_{1, I} | f_{1, I, FE} | I_{1, I, FE} | f_{1, exp} | I_{1, I, exp} | I_{1, I, cal} |
| (days) | (Hz) | (mm⁴) | (mm⁴) | (Hz) | (mm⁴) | (Hz) | (mm⁴) | (mm⁴) |
| 288, 290 | 15.32 | 1.43209 × 10⁹ | 1.44167 × 10⁹ | 15.17 | 1.44879 × 10⁹ | 15.60 | 1.54285 × 10⁹ | 1.43485 × 10⁹ |
| 291 | | 1.43211 × 10⁹ | 1.44167 × 10⁹ | | 1.44879 × 10⁹ | 15.62 | 1.54680 × 10⁹ | 1.43853 × 10⁹ |

[Identification of Prestress Forces]

Firstly, equation (4) is the formula of the magnification factor as follows:

$${v}_{t o t , mid} d = \frac{{v}_{I , mid}}{1 - {N}_{x} / {N}_{c r E}} , \qquad ( 4 )$$

wherein v_{tot,mid} is the static vertical deflection at the PCI girder-bridge's midspan; v_{I,mid} is the corresponding first-order static vertical deflection; Nₓ is the existing prestress force; whereas N_{crE} is the PC girder-bridge's Euler buckling load. Equation (4) is then transformed into equation (5) with simple manipulations:

$${N}_{x} = {N}_{crE} ( 1 - \frac{{v}_{I , mid}}{{v}_{tot , mid}} ) . \qquad ( 5 )$$

A first-order static vertical deflection v_{I}(x) along a single-span PCI girder-bridge can be determined by equation (6):

v_{I}(x)=(ψ/12)×(x/L)[¾−(x/L)²] (6).

v_{I,mid}=ψ/48 is gained by substituting x=L/2 into equation (6), wherein the loading parameter ψ is expressed by equation (V):

$$ψ = \frac{{FL}^{3}}{{E}_{exp , c , t} I} . \qquad ( V )$$

The Euler buckling load of a single-span PCI girder-bridge is calculated by equation (7):

$${N}_{c r E} = {π}^{2} \frac{{E}_{exp , c , t} I}{{L}^{2}} . \qquad ( 7 )$$

The non-dimensional prestress force (nₓ) is instead calculated by equation (8):

$${n}_{x} = \frac{{N}_{x} {L}^{2}}{{E}_{exp , c , t} I} . \qquad ( 8 )$$

Equation (I) for the non-dimensional prestress force (nₐ) was obtained by substituting equation (5), v_{I}=ψ/48, equation (V), and equation (7) into equation (8).

The prestress force (Nₐ) can consequently be identified by substituting nₐ into equation (II), which is transformed from equation (8).

At last, the prestress force (Nₐ) is identified by substituting the initial tangent Young's modulus (E_{exp,c, t}); the cross-sectional second moment of area obtained from different procedures, including I_{1,I} from the analytical solution, I_{1,I,FE} from the FE model, and I_{1,I,cal} from free bending vibration tests and subsequent calibration; and the static vertical deflection (v_{tot,mid}) measured with the three-point bending test into equation (I) and equation (II). The results were shown in Table 4, wherein the identifications were obtained assuming the initial tangent Young's modulus (E_{exp,c, t}) and the vertical deflections (v₄) measured at the PC girder-bridge's midspan (FIG. 19).

TABLE 4

| Age of | | | | | | | | | | | |
|---|---|---|---|---|---|---|---|---|---|---|---|
| prestressing | N_{x, aver} | F | | Nₐ | Δ | | N_{a, FE} | Δ_{FE} | | N_{a, cal} | Δ_{cal} |
| (days) | (kN) | (kN) | nₐ | (kN) | (%) | n_{a, FE} | (kN) | (%) | n_{a, cal} | (kN) | (%) |
| 288 | 516 | 23.2 | 0.42 | 466 | −9.7 | 0.47 | 519 | 0.6 | 0.38 | 414 | −19.8 |
| | | 34.2 | 0.44 | 480 | −7.0 | 0.48 | 534 | 3.5 | 0.39 | 429 | −16.9 |
| | | 42.2 | 0.46 | 502 | −2.7 | 0.50 | 556 | 7.8 | 0.41 | 451 | −12.6 |
| 290 | 516 | 24.2 | 0.35 | 383 | −25.8 | 0.39 | 437 | −15.3 | 0.30 | 332 | −35.7 |
| | | 33.2 | 0.41 | 446 | −13.2 | 0.45 | 501 | −2.9 | 0.36 | 396 | −23.3 |
| | | 42.8 | 0.49 | 535 | 3.7 | 0.53 | 588 | 14.0 | 0.44 | 483 | −6.4 |
| | | 51.4 | 0.50 | 556 | 7.8 | 0.55 | 610 | 18.2 | 0.46 | 504 | −2.3 |
| 291 | 515 | 26.5 | 0.29 | 325 | −36.9 | 0.34 | 378 | −26.6 | 0.27 | 301 | −41.6 |
| | | 34.1 | 0.34 | 375 | −27.2 | 0.39 | 429 | −16.7 | 0.32 | 351 | −31.8 |
| | | 42.1 | 0.43 | 473 | −8.2 | 0.48 | 526 | 2.1 | 0.41 | 449 | −12.8 |
| | | 51.9 | 0.52 | 573 | 11.3 | 0.57 | 626 | 21.6 | 0.50 | 549 | 6.6 |

In summary, the method for identifying prestress force in single or multi-span PCI girder-bridges, provided by the present invention, can be performed without causing any structural damage along the PCI bridge. Notably, the structural damage of drilling cores for measuring the initial tangent Young's modulus (E_{exp,c, t}), when it is necessary, is not serious. The prestress losses can then precisely be predicted through free bending vibration and three-point bending tests. Thus, the cost of identifying prestress force is significantly decreased.

The aforementioned laboratory simulations were intended to illustrate the embodiments of the subject invention and the technical features thereof, but not for restricting the scope of protection of the subject invention. Other possible modifications and/or variations can be made without departing from the spirit and scope of the invention as hereinafter claimed. Particularly, this is referred to the analytical and experimental evaluation of the initial tangent Young's modulus of concrete at the time of testing, and to the assumption of different geometrical properties and boundary conditions along the PCI girder-bridges. The scope of the subject invention is based on the claims as appended.

What is claimed is:

1. A method for identifying prestress force in single-span or multi-span PCI girder-bridges, comprising the steps of:
(A) obtaining a total length (L) and a first-order fundamental frequency (f_{1,I}) of a PCI girder-bridge, and calculating or measuring an initial tangent Young's modulus (E_{exp,c,t}) and a cross-sectional second moment of area (I_{1,I}) of the PCI girder-bridge;
(B) performing a three-point bending test through a vertical load (F) for measuring a static vertical deflection at the PCI girder-bridge's midspan (v_{tot,mid}) and a loading parameter (ψ);
(C) calculating a non-dimensional prestress force (nₐ) by an equation (I):
$${n}_{a} = {π}^{2} ( 1 - \frac{ψ}{𝒳 {v}_{tot , mid}} ) {x}^{2} ; \qquad ( I )$$
wherein x is 1 and χ is 48 when the PCI girder-bridge is a single span of length L; x is 2 and χ is 534.26 when the PCI-girder-bridge is an equidistant two-span of length L; x is 3 and χ is 2356.35 when the PCI-girder-bridge is an equidistant three-span of length L; and
(D) determining the prestress force (Nₐ) by an equation (II):
$${N}_{a} = \frac{{E}_{exp , c , t} I}{{L}^{2}} {n}_{a} . \qquad ( II )$$

2. The method of claim 1, wherein step (A), when the initial tangent Young's modulus (E_{exp,c, t}) and the PCI girder-bridge's total self-mass per unit length (m_{PCI+d}) are known, the first-order fundamental frequency (f_{1,I}) is evaluated.

3. The method of claim 2, wherein step (A), when the PCI girder-bridge is the single-span of length L, the first-order fundamental frequency (f_{1,I}) is calculated by an analytical solution, the cross-sectional second moment of area (I_{1,I}) is calculated by an equation (III-1) based on Euler-Bernoulli theory:
$${I}_{1 , I} = \frac{4 {f}_{1 , I}^{2} {m}_{PCI + d} {L}^{4}}{{π}^{2} {E}_{exp , c , t} g} ; \qquad ( III - 1 )$$
wherein g=9.81 m/s².

4. The method of claim 3, wherein the first-order fundamental frequency (f_{1,I}) is calculated by the analytical solution shown in equation (2):
$${f}_{1 , I} = \sqrt{\frac{{E}_{exp , c , t} {I}_{tot , mid} {π}^{5} + 32 λ {f}_{t} {L}^{2}}{2 {m}_{PCI + d} π {L}^{4}}} ; \qquad ( 2 )$$
wherein I_{tot,mid} is the cross-sectional second moment of area of the PCI girder-bridge's midspan; λ is a first-order coefficient, fₜ is a deflected shape of a parabolic tendon.

5. The method of claim 4, wherein the first-order coefficient λ is calculated by equation (3):
$$λ = \frac{{E}_{t} {A}_{t}}{{L}_{t}} [ \frac{16 {f}_{t}}{π L} - \frac{2 {L}^{3}}{{E}_{exp , c , t} {I}_{tot , mid} {π}^{3}} ( - {m}_{PCI + d} ) ] ; \qquad ( 3 )$$
wherein Eₜ is a Young's modulus of the parabolic tendon; Aₜ is a cross-sectional area of the parabolic tendon; Lₜ is an effective length of the parabolic tendon.

6. The method of claim 2, wherein step (A), when the PCI girder-bridge is single or multi-span, the first-order fundamental frequency (f_{1,I,FE}) is calculated by a Finite Element (FE) model, the cross-sectional second moment of area (I_{1,I,FE}) is consequently determined based on Euler-Bernoulli theory; when the PCI girder-bridge is the single-span of length L, the cross-sectional second moment of area (I_{1,I,FE}) is calculated by equation (III-2-1):
$${I}_{1 , I , FE} = \frac{4 {f}_{1 , I , FE}^{2} {m}_{t o t} {L}^{4}}{{π}^{2} {E}_{exp , c , t} g} ; \qquad ( III - 2 - 1 )$$
when the PCI girder-bridge is the equidistant two-span of length L, the cross-sectional second moment of area (I_{1,I,FE}) is calculated by equation (III-2-2):
$${I}_{1 , I , FE} = \frac{{f}_{1 , I , FE}^{2} {m}_{t o t} {L}^{4}}{4 {π}^{2} {E}_{exp , c , t} g} ; \qquad ( III - 2 \text{-2)}$$
and
when the PCI girder-bridge is the equidistant three-span of length L, the cross-sectional second moment of area (I_{1,I,FE}) is calculated by equation (III-2-3):
$${I}_{1 , I , FE} = \frac{{f}_{1 , I , FE}^{2} {m}_{t o t} {L}^{4}}{20.25 {π}^{2} {E}_{exp , c , t} g} ; \qquad ( III - 2 - 3 ) ;$$
wherein equations (III-2-1) to (III-2-3), g=9.81 m/s², mₜₒₜ is the PCI girder-bridge's total self-mass per unit length, whereas I_{1,I,FE} is regarded to the cross-sectional second moment of area (I_{1,I}) for subsequent steps;
wherein eccentricities of parabolic tendon e₁ and e₂, or e₁, e₂ and e₃ are considered in the FE models.

7. The method of claim 1, wherein step (A), when the cross-sectional second moment of area (I_{1,I}) of the PCI girder-bridge is unknown, and when the PCI girder-bridge is multi-span or single-span, the first-order fundamental frequency (f_{1,exp}) is measured through free bending vibration tests, whereas the cross-sectional second moment of area (I_{1,I,exp}) is calculated based on the Euler-Bernoulli theory:
when the PCI girder-bridge is the single-span of length L, the cross-sectional second moment of area (I_{1,I,exp}) is calculated by equation (III-3-1):
$${I}_{1 , I , exp} = \frac{4 {f}_{1 , exp}^{2} {m}_{t o t} {L}^{4}}{{π}^{2} {E}_{exp , c , t} g} ; \qquad ( III - 3 \text{-1)}$$
when the PCI girder-bridge is the equidistant two-span of length L, the cross-sectional second moment of area (I_{1,I,exp}) is calculated by equation (III-3-2):
$${I}_{1 , I , exp} = \frac{{f}_{1 , exp}^{2} {m}_{t o t} {L}^{4}}{4 {π}^{2} {E}_{exp , c , t} g} ; \qquad ( III - 3 \text{-2} )$$
and
when the PCI girder-bridge is the equidistant three-span of length L, the cross-sectional second moment of area (I_{1,I,exp}) is calculated by equation (III-3-3):
$${I}_{1 , I , exp} = \frac{{f}_{1 , exp}^{2} {m}_{t o t} {L}^{4}}{20.25 {π}^{2} {E}_{exp , c , t} g} ; \qquad ( III - 3 - 3 )$$
wherein equations (III-3-1) to (III-3-3), g=9.81 m/s²; mₜₒₜ is the PCI girder-bridge's total self-mass per unit length;
a calibrated cross-sectional second moment of area (I_{1,I,cal}) is consequently calculated by an equation (IV):
I_{1,I,cal}=0.93×I_{1,I,exp} (IV);
wherein the calibrated cross-sectional second moment of area (I_{1,I,cal}) is regarded as the cross-sectional second moment of area (I_{1,I}) for subsequent steps.

8. The method of claim 1, wherein step (B), the loading parameter (y) is measured by an equation (V):
$$ψ = \frac{{FL}^{3}}{{E}_{exp , c , t} I} . \qquad ( V )$$
